SEQUENCE LISTING (1 sequence)

SEQ ID NO: 1 - DNA, 354 bp, synthetic construct
  source 1..354: mol_type = other DNA
aacgtttgttacgaacttcaccccagtcatgaatcacaccgtggtaaccgtcctcccgaaggttagattatctacttctggtgcaacccactcccatggtgtgacgggcggtgtgtacaaggcccgggaacgtattcaccgcgacattctgattcgcgattactagcgattccgacttcacgcacgtcgagttgcagactgcgatccggactacgatcggttttgtgagattagctccacctcgcggcttggcgaccctctgtaccgaccattgtagcacgtgtgtagcccaggccgtaagggccatgatgacttgacgtcatccccaccttcctccggtttgtcaccggcagt

(19) United States
(12) Patent Application Publication
(10) Pub. No.: US 2025/0207219 A1
(43) Pub. Date: Jun. 26, 2025

(54) The Use of Cyanogenic Bacteria to Leach Gold

(71) Applicant: National Research Council of Canada, Ottawa (CA)

(72) Inventor: Nancy Perreault, Montréal (CA)

(73) Assignee: National Research Council of Canada, Ottawa, ON (CA)

(21) Appl. No.: 18/846,424

(22) PCT Filed: Mar. 17, 2023
(86) PCT No.: PCT/CA2023/050346

§ 371 Date: Sep. 12, 2024

(30) Foreign Application Priority Data
Mar. 18, 2022 (CA) 3152732

(51) Int. Cl. C22B 11/00; C12N 1/20; C12P 13/00; C12R 1/38

(52) U.S. Cl. CPC C22B 11/04; C12N 1/205; C12P 13/002; C22B 11/00; C12R 2001/38

(57) ABSTRACT

The present invention concerns new strains of cyanogenic bacteria, and more particularly alkali-tolerant Pseudomonas bacteria, that are useful to gold mining activities. These bacteria are useful to leach gold from any source including ores, concentrates or waste material. They have the ability to effectively produce cyanide at alkaline pH.

### TECHNICAL FIELD

New strains of bacteria were discovered, cyanogenic bacteria, and more particularly alkali-tolerant Pseudomonas bacteria. These bacteria are useful in metallurgical processes that requires cyanidation, such as, for example, gold mining activities.

The present invention concerns the use of cyanogenic bacteria, and more particularly the use of alkali-tolerant Pseudomonas bacteria, for leaching gold from any source including ores, concentrates or waste materials.

### BACKGROUND

Chemical cyanidation at alkaline pH is the most common process used in metallurgy to extract gold from ores and concentrates. This technique relies on the property of cyanide to form soluble and chemically stable complexes with gold. Chemical cyanidation is carried out at a pH greater than 10.3 to maximize the concentration of cyanide (CN—) and to prevent its volatilization in the form of hydrogen cyanide (HCN). Efficient extraction requires from 300 to 2,000 g of sodium cyanide per tonne of ore. Cyanidation residues produce highly alkaline sludge rich in CN—, in stable metal cyanide complexes and cyanide transformation products. The impact of cyanidation on the environment, particularly on aquatic species, is well documented. Several alternatives to the cyanidation process have been proposed. However, despite the costs associated with cyanidation, safety related to cyanide toxicity and adverse environmental effects, this method remains the most effective to date to meet high production targets. In recent years, the idea of using cyanogenic bacteria, i.e., that produce cyanide, has emerged as an alternative to the chemical leaching of gold. Most studies on bio-cyanidation have focused on understanding the natural phenomenon (e.g., weathering) and, more recently, on gold leaching from e-waste. The concept of bio-cyanidation is still at the low technology readiness level and the objective of this R&D effort was to evaluate if it could be applied to mining.

Biocyanidation, the production of cyanide (more specifically HCN) by microorganism, has been observed in several organisms (bacteria, fungi, algae and certain plants). The majority of the cyanogenic bacteria known so far are Pseudomonas, mainly strains of P. aeruginosa and P. fluorescens. Other cyanogenic bacteria include Chromobacterium violaceum, Rhizobium leguminosarum, as well as species of Bacillus, Burkholderia and cyanobacteria. Cyanide is produced as a secondary metabolite and peaks at the beginning of the stationary growth phase (Castric 1975). It is synthesized from glycine by oxidative decarboxylation, reaction catalyzed by the enzyme HCN synthase (Castric 1977) encoded by the operon hcnABC. Unlike the acidophilic bacteria generally used in biohydrometallurgy, such as Acidithiobacillus ferrooxidans, cyanogenic bacteria mobilize metals in neutral or slightly alkaline conditions.

### SUMMARY

The Applicant has discovered an alkali-tolerant Pseudomonas bacteria having the ability to produce cyanide. The AE-27 strain which was deposited at the International Depositary Authority of Canada (IDAC) on Mar. 16, 2022, under Accession Number 160322-01 is phylogenetically related to Pseudomonas putida.

Advantageously, the AE-27 stain can be used in biocyanidation processes for leaching gold or other precious metals. The cyanide-containing solution produced by the AE-27 strain may complement or replace chemical cyanide used in conventional metallurgical processes.

The present disclosure therefore relates in one aspect thereof to an alkali-tolerant Pseudomonas bacteria having the ability to produce cyanide.

In some embodiments, the Pseudomonas bacteria is capable of leaching gold.

In some embodiments, the Pseudomonas bacteria is characterized by a 16S rRNA sequence at least 90% identical, at least 95% identical, at least 96% at least 97%, at least 98%, at least 99% identical to the sequence set forth in SEQ ID NO: 1 or identical to SEQ ID NO: 1.

SEQ ID NO: 1: 16S rRNA sequence

AACGTTTGTTACGAACTTCACCCCAGTCATGAATCACACCGTGGTAACC
GTCCTCCCGAAGGTTAGATTATCTACTTCTGGTGCAACCCACTCCCATG
GTGTGACGGGCGGTGTGTACAAGGCCCGGGAACGTATTCACCGCGACA
TTCTGATTCGCGATTACTAGCGATTCCGACTTCACGCACGTCGAGTTGC
AGACTGCGATCCGGACTACGATCGGTTTTGTGAGATTAGCTCCACCTCG
CGGCTTGGCGACCCTCTGTACCGACCATTGTAGCACGTGTGTAGCCCAG
GCCGTAAGGGCCATGATGACTTGACGTCATCCCCACCTTCCTCCGGTTT
GTCACCGGCAGT

More particularly, the present disclosure relates in one aspect thereof to strain AE-27 deposited at International Depositary Authority of Canada on Mar. 16, 2022, under Accession Number 160322-01.

In another aspect, the present disclosure relates to the use of the bacteria disclosed herein for producing cyanide.

In yet another aspect, the present disclosure relates to the use of the bacteria disclosed herein for producing a lixiviant.

In another aspect, the present disclosure relates to the use of the bacteria disclosed herein in a cyanide leaching process.

In some embodiments, the cyanide leaching process of the present disclosure (biocyanidation) is for extracting precious metal from material. The precious metal may be, for example and without limitations, gold and/or silver or another metal that is able to form a complex with cyanide ions.

The cyanide leaching process may be applicable to any source of material that may potentially contain precious metals.

In exemplary embodiments, the source of material is ores. In a particular embodiment, the source of material is gold ores. In exemplary embodiments, the gold ore is non-refractory gold ore or pre-treated refractory gold ore.

In another exemplary embodiment, the source of material is a concentrate. In an exemplary embodiment, the concentrate contains gold.

In another exemplary embodiment, the source of material is waste material. An exemplary embodiment of waste material is electronic waste.

In another aspect, the present disclosure relates to a process for leaching precious metals from ore, concentrate or waste material using the Pseudomonas bacteria disclosed herein or a preparation thereof as a lixiviant.

In yet another aspect, the present disclosure relates to a process for obtaining precious metals from ore, concentrate or waste material that comprises contacting the ore, concentrate or waste material with the Pseudomonas bacteria disclosed herein or with a preparation thereof and isolating the precious metal. The process may be carried under conditions suitable for production of cyanide by the bacteria.

In exemplary embodiments, the process is carried out to obtain gold and/or silver.

In exemplary embodiments, the process is performed at an alkaline pH. In some exemplary embodiments, the pH may be, for example, between 8.0 and 10.0. In other exemplary embodiments, the pH may be between 8.5 and 9.5. In yet other exemplary embodiments, the pH may be above 8.0. In other exemplary embodiments, the pH is above 8.5. In further exemplary embodiments, the pH is approximately 9.0.

In exemplary embodiments the process is performed at a temperature of between approximately 4° C. to approximately 35° C. For example, the process may be performed at a temperature of between 4° C.±2° C. to 35° C.±2° C.

In other exemplary embodiments, the process is performed at a temperature of between approximately 25° C. to approximately 35° C. For example, the process may be performed at a temperature of between 25° C.±2° C. to 35° C.±2° C.

In an exemplary embodiment, the process is performed at a temperature of approximately 25° C. For example, the process may be performed at a temperature of 25° C.±2° C.

In exemplary embodiments, the process may be performed in a medium that allows production of cyanide by the bacteria. In some embodiments, the medium is an aqueous solution. In other embodiments, in the medium is a mineral medium. In some embodiments, the medium includes a substrate for the hydrogen cyanide synthase enzyme. The medium may also comprise a buffer, nutrients, a source of energy, and/or minerals.

An exemplary embodiment of a substrate for the hydrogen cyanide synthase enzyme includes glycine. Threonine, phenylalanine as well as other amino acids may represent alternative substrates.

In exemplary embodiments, the medium comprises glycine. The concentration of glycine may be from about 1 g/L to about 10 g/L. In some embodiments, glycine may be at a concentration of from about 2.5 g/L to about 7.5 g/L. In an exemplary embodiment, glycine is at a concentration of approximately 5 g/L.

In some embodiments, nutrients may be added to the medium at the start of the process. However, nutrients may be gradually added to the medium. In some embodiments, at least a quantity of the nutrients is added at the beginning of the process. In other embodiments, at least a quantity of nutrients is added during the process.

In some embodiments, the process is a batch process. In other embodiments, the process is a fed-batch process. In other embodiments, the process is a continuous culture process.

Accordingly, in some embodiments, glycine may be added to the medium at the start of the process. Alternatively, glycine may be gradually added to the medium. For example, at least a quantity of glycine is added at the beginning of the process. In other example, at least a quantity of glycine is added during the process.

In accordance with the present disclosure, cyanide production does not appear to be significantly affected by pulp density from about 1% to about 5%.

In some embodiments, the process is performed at a pulp density of from about 1% to at least about 20%, from about 1% to about 20%, from about 1% to about 10%, from about 1% to about 5% or any other ranges therebetween. In exemplary embodiments, the process is carried out at pulp density of at least 1%, at least 5% or higher than 5%.

In some embodiments, the process is performed with ore concentration ranging from about 1 g/L to at least about 200 g/L, from about 10 g/L to at least about 200 g/L, from about 50 g/L to at least about 200 g/L, from about 100 g/L to at least about 200 g/L, from about 150 g/L to at least about 200 g/L, or any other ranges therebetween.

In accordance with the present disclosure, the process may be performed in a mineral medium comprising glycine at a concentration of approximately 5 g/L, at a pH of approximately 9.0, and at a temperature of approximately 25° C.

In some embodiment the process is performed in a medium having a salinity of from bout 0% to about 6% NaCl. In some embodiment the process is performed in a medium having a salinity of from about 0.5% to about 6% NaCl. In some embodiment the process is performed in a medium having a salinity of up to about 6% NaCl. In some embodiment the process is performed in a medium having a salinity of at least about 6% NaCl.

The process of the present disclosure may preferably be carried out during the growth phase of the bacteria. In some embodiments, the process of the present disclosure the process is carried out during cyanide production by the bacteria. In some embodiments, the process of the present disclosure the process is carried out until at least maximum cyanide production is achieved.

In accordance with the present disclosure, the process is carried out in a mineral medium. In some instance, the mineral medium may comprise for example, potassium, sodium, magnesium, and/or iron. In exemplary embodiments, the mineral medium may comprise for example, K₂HPO₄, NaH₂PO₄, MgSO₄, ferric citrate. In some embodiments, the mineral medium may further comprise amino acids. In exemplary embodiments, the mineral medium comprises glycine. In other exemplary embodiments, the mineral medium comprises L-methionine and/or L-glutamic acid.

In some embodiments, the mineral medium comprises NaCl. In some embodiments, the mineral medium comprises glucose.

In accordance with the present disclosure, the mineral medium may comprise approximately 5 mM K₂HPO₄, approximately 5 mM NaH₂PO₄, approximately 2 mM MgSO₄·H₂O, approximately 50 mM Tris, 0.02 mM ferric citrate, approximately 12.5 mM glycine, approximately 5 mM L-methionine and approximately 20 mM L-glutamic acid.

In accordance with the present disclosure, the mineral medium may comprise approximately 22 mM KH₂PO₄, approximately 48 mM Na₂HPO₄, approximately 2 mM MgSO₄·7H₂O, approximately 0.1 mM NaCl, approximately 50 mM Tris, 6.70 mM L-methionine, approximately 22 mM glucose, approximately 20 mM L-glutamic acid and approximately 0.02 mM ferric citrate and glycine at a concentration of from about 1 g/L to about 10 g/L.

The present disclosure also relates to a cyanide-containing solution obtained from the bacteria described herein. In some embodiments, the cyanide-containing solution may be substantially-free of bacteria.

The cyanide-containing solution may be obtained by growing bacteria under conditions suitable for production of cyanide by the bacteria. In some embodiments, the bacteria may be removed from the cyanide-containing solution. In other embodiments, the bacteria may be inactivated within the cyanide-containing solution. In yet another embodiment, the bacteria may be left in the cyanide-containing solution.

Exemplary conditions for obtaining a cyanide-containing solution are disclosed herein. In some embodiments, the bacteria is able to produce cyanide at a concentration of at least 1 mg/L, at least 5 mg/L, at least 10 mg/L, at least 15 mg/L or higher than 15 mg/L.

The present disclosure therefore relates to the use of the cyanide-containing solution described herein as a lixiviant.

In some embodiments, the cyanide-containing solution may be used in a cyanide leaching process. The leaching process may be carried out by a method that comprises contacting the ore, concentrate or waste material with the cyanide-containing solution.

The present disclosure therefore relates to a process for obtaining precious metals from ore, concentrate or waste material that comprises contacting the ore, concentrate or waste material with a cyanide-containing solution obtained from Pseudomonas bacteria disclosed herein and isolating the precious metal. In some embodiments, the precious metal is isolated from a pregnant solution or slurry.

The present disclosure also provides a product obtained by the process disclosed herein. In some embodiments, the product comprises precious metals such as for example and without limitations, gold and/or silver.

The present disclosure also relates to a kit comprising one or more containers comprising the alkali-tolerant Pseudomonas bacteria described herein. Instructions for amplifying and/or using the bacteria may also be included.

In some embodiment, the kit comprises one or more container for the alkali-tolerant Pseudomonas bacteria described herein and one or more containers comprising growth medium (or separated components for making medium).

In some embodiment, the kit comprises one or more container for the alkali-tolerant Pseudomonas bacteria described herein and one or more containers comprising medium (or separated components for making medium) suitable for production of cyanide by the bacteria.

The present disclosure also relates to a piece of equipment for mining containing the alkali-tolerant Pseudomonas bacteria described or the cyanide-containing solution described herein.

### BRIEF DESCRIPTION OF THE DRAWING

FIG. 1: Screening of bacteria isolated from the AEM samples for cyanide production using the 0.5% picrate/2% Na2CO3 technique. Color change from yellow (light grey) to orange/red (darker grey) is indicative of HCN production. Strain Pf-5 was used as a positive control. AE-27 and AE-45 were later shown to be the same strain.

FIG. 2: Tolerance of AE-27 to different ore concentrations

FIG. 3: Kinetics of cyanide production by strains AE-27 and Pf-5 at pH 8.5.

FIG. 4: Cyanide production (mg CN/L) as a function of glycine concentration at 2% pulp density

FIG. 5: Cyanide production (mg CN/L) as a function of pH at 2% pulp density

FIG. 6: Cyanide production (mg CN/L) as a function of pulp density

FIG. 7: Cyanide production (mg CN/L) as a function of temperature at 2% pulp density.

### DETAILED DESCRIPTION

### Definitions

The use of the terms “a” and “an” and “the” and similar referents in the context of describing embodiments (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”, and each specific combination.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

The term “about” or “approximately” with respect to a given value means that variation in the value is contemplated. In some embodiments, the term “about” or “approximately” shall generally mean a range within +/−10 percent, within +/−5 percent, within +/−4 percent, within +/−3 percent, within +/−2 percent or within +/−1 percent of a given value or range.

It is to be understood herein, that expressions referring to ranges of values in the format such as “from A to B”, include each individual value and any sub-range comprised and including such ranges. For example, the expression “from 1 to 10” includes sub-ranges such as and without limitations, “from 2 to 10”, “from 2 to 9”, “from 3 to 6”, “from 5 to 7” and any individual values comprised between and including 1 and 10, i.e., 1, 2, 3, 4, 5, 6, 7, 8, 9 or 10.

It is to be understood herein that the term “at least” with respect to a given value intends to include the value and superior values. For example, the term “at least 80%” includes “at least 80%”, “at least 81%”, “at least 82%”, “at least 83%”, “at least 84%”, “at least 85%”, “at least 86%”, “at least 87%”, “at least 88%”, “at least 89%”, “at least 90%”, “at least 91%”, “at least 92%”, “at least 93%”, “at least 94%”, “at least 95%”, “at least 96%”, “at least 97%”, “at least 98%”, “at least 99%”, “at least 99.1%”, “at least 99.2%”, at least 99.3% “, at least 99.4%”, at least 99.5% “, at least 99.6%”, at least 99.7% “, at least 99.8%”, at least 99.9%”, and 100%.

The term “exemplary” as used herein refers to “serving as an example, instance, or illustration,” and not “preferred” or “advantageous over other embodiments”.

### Pseudomonas Bacteria Producing Cyanide and Preparation Thereof

New bacteria were found to show capability to leach gold from crushed ore. The cyanide-producing bacterium was isolated from mining samples and phylogenetically (16 rRNA gene sequence) identified as a Pseudomonas strain. Alkali-tolerant bacteria were isolated from samples collected at various mine sites. Among the 70 bacterial isolates screened, two were shown to produce cyanide (isolates AE-27 and AE-45). Both strains were phylogenetically closely related to Pseudomonas putida. AE-27 and AE-45 were later shown to be the same strain, and therefore, strain AE-27 was deposited at IDAC (International Depositary Authority of Canada) on Mar. 16, 2022, and was assigned the Accession Number 160322-01.

Strain AE-27 grows at pH up to 9.5, at temperature from 4° C. to 37° C., and salt concentration (NaCl) up to 6%. Strain AE-27 grows in the presence of high ore concentrations with no observable toxicity at the highest concentration tested (500 g/L). Among the conditions tested, cyanide production by AE-27 is optimal in a mineral medium adjusted at pH 9.0, containing glycine (as precursor of cyanide) at a concentration of 5 g/L, and incubated at 25° C. AE-27 could produce 16 mg free CN/L at pH 9.0 without fine optimization. Gold extraction by AE-27 has reached 14% after 48 h of incubation at 5% ore pulp density at pH 9.0 when incubated at room temperature (22° C.).

AE-27 grew at pH up to 9.5, at temperature up to 37° C., and salt concentration (NaCl) up to 6%, which was slightly better than what was observed for a known cyanogenic strain, Pseudomonas protegens Pf-5 (ATCC BAA477), which did not grow at pH 9.0 and above. Strain AE-27 could grow in the presence of high ore concentrations with no observable toxicity at the highest concentration tested (500 g/L). Parameters that can influence cyanide production were evaluated (glycine concentration in the culture medium, pH, ore pulp density, temperature) in shake flasks. Results showed an optimal glycine concentration of 5 g/L, at a pH of 9.0, and temperature of 25° C. AE-27 could produce 16 mg free CN/L at pH 9.0 without optimization. Gold extraction by AE-27 reached 14% after 48 h of incubation at 5% pulp density at pH 9.0 at room temperature (˜22° C.). Gold in solution was below the ICP-MS detection limit when incubation was prolonged to 120 h in an attempt to solubilize more gold. Biodegradation of the cyanide-Au complex was suspected in this case.

Overall, the results showed that bacteria isolated as part of this project successfully dissolved gold from a crushed ore sample at relatively high pH without any adaptation stage. Efforts will now focus to extend the biocyanidation period over 48 h and improve cyanide yields.

The present disclosure provides an alkali-tolerant Pseudomonas bacteria having the ability to produce cyanide. The alkali-tolerant Pseudomonas bacteria is also capable of leaching gold. Moreover, the alkali-tolerant Pseudomonas bacteria is capable of leaching silver.

In some embodiments, the Pseudomonas bacteria is the AE-27 strain which was deposited at the International Depositary Authority of Canada (IDAC) on Mar. 16, 2022, under Accession Number 160322-01.

In some embodiments, the Pseudomonas bacteria is characterized as having a 16S rRNA sequence that is at least 90% identical, at least 95% identical, at least 99% identical to the sequence set forth in SEQ ID NO: 1 or identical to SEQ ID NO:1.

The conditions for conservation of bacteria are well known to a person skilled in the art and includes without limitations, lyophilisation, freezing with cryoprotectants (e.g., −150° C. (liquid nitrogen), −80° C., −20° C.), solutions (e.g., 4° C.) and agar plates (4° C.).

The Pseudomonas bacteria of the present disclosure may be provided in solution.

Pseudomonas bacteria of the present disclosure may be conserved at various temperatures ranging from at least −80° C. to at least 37° C.

A preparation of the Pseudomonas bacteria refers to a solution that contains a sufficient quantity of bacteria for carrying out the process described herein.

### Growth Conditions

The present disclosure also relates to a preparation of bacteria that comprises bacterial medium for growth or maintenance including for example and without limitation Luria Broth (LB), Terrific Broth (TB), M9 minimal, or another medium known to a person skilled in the art.

For example, bacteria are inoculated in vessels containing growth medium (e.g., LB or modified LB) in shaking incubator (150 rpm) at temperature ranging for example, from 20° C. to 30° C.

Small scale growth may be performed in shaker flasks, whereas large-scale growth will entail growing the bacteria in bioreactors.

### Conditions for Cyanide Production

The Pseudomonas bacteria of the present disclosure expresses hydrogen cyanide synthase enzymes that convert amino acids, such as glycine, into hydrogen cyanide. The Pseudomonas bacteria of the present disclosure may thus produce cyanide.

To that effect, the bacteria are grown in a medium that comprises a substrate of the hydrogen cyanide synthase enzyme. In some embodiments, the medium is an aqueous solution that may comprise a buffer, nutrients, a source of energy, and/or minerals and a substrate of the hydrogen cyanide synthase enzyme. In some embodiments, the medium is a mineral medium. In some instances, the starting material may contain sufficient minerals such that no external source of minerals is needed.

In some embodiments, the buffer may comprise a phosphate buffer.

In some embodiments, the nutrients may comprise a source of amino acids, peptides, proteins, and combination thereof and optionally vitamins. Therefore, in exemplary embodiments the medium may comprise L-methionine and/or L-glutamic acid. In another embodiments the medium may comprise tryptone, peptone, polypeptone and/or yeast extract.

In some embodiments, the minerals comprise one or more elements selected from the group consisting of calcium, phosphorus, sodium, potassium, magnesium, manganese, sulfur, chloride, iron, iodine, fluoride, zinc, copper, selenium, chromium, cobalt and combination thereof.

In an exemplary embodiment, the minerals comprise magnesium, iron, sodium, potassium.

In some embodiments, the source of energy may comprise sugars. In an exemplary embodiment, the source of energy is glucose. In another exemplary embodiment, the source of energy is fructose. In some embodiments, the source of energy may come from food waste, plant waste, animal waste and the like.

In some embodiments, the substrate of the hydrogen cyanide synthase enzyme is glycine. In other embodiments, the substrate of the hydrogen cyanide synthase enzyme is threonine. In yet other embodiments, the substrate of the hydrogen cyanide synthase enzyme is phenylalanine.

In some embodiments, the concentration of glycine in the medium may be from about 1 g/L to about 10 g/L. For instances, glycine may be at a concentration of from about 2.5 g/L to about 5.0 g/L, at a concentration of from about 2.5 g/L to about 7.5 g/L, at a concentration of from about 5.0 g/L to about 7.5 g/L.

In an exemplary embodiment, glycine is at a concentration of approximately 1 g/L, approximately 2 g/L, approximately 2.5 g/L, approximately 3 g/L, approximately 3.5 g/L, approximately 4 g/L, approximately 4.5 g/L, approximately 5 g/L, approximately 5.5 g/L, approximately 6 g/L, approximately 6.5 g/L, approximately 7 g/L, approximately 7.5 g/L, approximately 8 g/L, approximately 8.5 g/L, approximately 9 g/L, approximately 9.5 g/L, approximately 10 g/L or higher than 10 g/L such as approximately 10.5 g/L, approximately 11 g/L, approximately 11.5 g/L, approximately 12 g/L.

The temperature, pH, salinity may also have an impact on the optimal production of cyanide.

The pH of the medium is adjusted to optimize cyanide production by the Pseudomonas bacteria. The Pseudomonas bacteria of the present disclosure produces cyanide under alkaline pH.

In some exemplary embodiments, the pH may be, for example, between 8.0 and 10.0. In other exemplary embodiments, the pH may be between 8.5 and 9.5. In yet other exemplary embodiments, the pH may be above 8.0. In other exemplary embodiments, the pH is above 8.5. In further exemplary embodiments, the pH is approximately 8.0. In other exemplary embodiments, the pH is approximately 8.5. In further exemplary embodiments, the pH is approximately 9.0. In other exemplary embodiments, the pH is approximately 9.5

The temperature is adjusted to optimize cyanide production by the Pseudomonas bacteria. In some embodiments, the temperature is between approximately 4° C. to approximately 35° C. In other exemplary embodiments, the temperature is between approximately 20° C. to approximately 35° C. In yet other exemplary embodiments, the temperature is between approximately 22° C. to approximately 37° C. In other exemplary embodiments, the temperature is between approximately 25° C. to approximately 37° C. In other exemplary embodiments, the temperature is between approximately 25° C. to approximately 35° C.

In an exemplary embodiment, the temperature may be between 4° C.±2° C. to 35° C.±2° C.

In another exemplary embodiment, the temperature may be between 20° C.±2° C. to 35° C.±2° C. In yet another exemplary embodiment, the temperature may be between 22° C.±2° C. to 37° C.±2° C. In a further exemplary embodiment, the temperature may be between 25° C.±2° C. to 37° C.±2° C. In yet a further exemplary embodiment, the temperature may be between 25° C.±2° C. to 35° C.±2° C.

In an exemplary embodiment, the temperature is approximately 4° C. In another exemplary embodiment, the temperature is approximately 20° C. In yet another exemplary embodiment, the temperature is approximately 22° C. In a further exemplary embodiment, the temperature is approximately 25° C. In yet a further exemplary embodiment, the temperature is approximately 27° C. In another exemplary embodiment, the temperature is approximately 30° C. In another exemplary embodiment, the temperature is approximately 35° C.

In accordance with an embodiment of the present disclosure the temperature may be 20° C.±2° C. In accordance with another embodiment of the present disclosure the temperature may be 22° C.±2° C. In accordance with another embodiment of the present disclosure the temperature may be 25° C.±2° C. In accordance with yet another embodiment of the present disclosure the temperature may be 27° C.±2° C. In accordance with a further embodiment of the present disclosure the temperature may be 30° C.±2° C. In accordance with yet a further embodiment of the present disclosure the temperature may be 35° C.±2° C.

In some embodiments, the Pseudomonas bacteria are grown in a mineral medium comprising glycine at a concentration of approximately 5 g/L and is at a pH of approximately 8.0. In other embodiments, the Pseudomonas bacteria are grown in a mineral medium comprising glycine at a concentration of approximately 5 g/L and is at a pH of approximately 8.5. In yet other embodiments, the Pseudomonas bacteria are grown in a mineral medium comprising glycine at a concentration of approximately 5 g/L and is at a pH of approximately 9.0.

The mineral medium may comprise potassium salt, sodium salt, magnesium salt, and/or iron salt. The mineral medium may comprise a source of amino acids and sugars.

In some embodiments, the mineral medium comprises NaCl.

The salinity of the medium may be for example, from about 0% to about 6% (w/v) NaCl. In some embodiment the salinity of the medium is from about 0.5% (w/v) to about 6% (w/v) NaCl.

In some embodiments the salinity of the medium is approximately 1.0%, approximately 1.5% (w/v), approximately 2.0% (w/v), approximately 2.5% (w/v), approximately 3.0% (w/v), approximately 3.5% (w/v), approximately 4.0% (w/v), approximately 4.5% (w/v), approximately 5.0% (w/v), approximately 5.5% (w/v), approximately 6.0% (w/v), approximately 6.5% (w/v) or approximately 7.0% (w/v) NaCl.

In some embodiment the salinity of the medium is up to about 6% (w/v) NaCl. In some embodiments the salinity of the medium is at least about 6% (w/v) NaCl.

In some embodiments, the mineral medium may comprise for example, K₂HPO₄, NaH₂PO₄, MgSO₄, and/or ferric citrate or equivalents thereof. In some embodiments, the mineral medium may further comprise amino acids. In exemplary embodiments, the mineral medium comprises L-methionine and/or L-glutamic acid. In other exemplary embodiments, the mineral medium may comprise glucose.

In some embodiments, the concentration of K₂HPO₄ in the medium is from about 0 to about 25 mM. In an exemplary embodiment, the concentration of K₂HPO₄ in the medium is from about 0 to about 9.4 mM. In yet other embodiments, the concentration of K₂HPO₄ in the medium is about 1 mM, about 5 mM, about 10 mM, about 15 mM, about 20 mM or about 25 mM.

In some embodiments, the concentration of NaH₂PO₄ in the medium is from about 0 to about 50 mM. In an exemplary embodiment, the concentration of NaH₂PO₄ in the medium is from about 0 to about 9.4 mM. In yet other embodiments, the concentration of NaH₂PO₄ in the medium is about 1 mM, about 5 mM, about 10 mM, about 15 mM, about 20 mM, about 25 mM, about 30 mM, about 35 mM, about 40 mM, about 45 mM, or about 50 mM.

In some embodiments, the concentration of MgSO₄ in the medium is from about 1 mM to about 3 mM. In some embodiments, the concentration of MgSO₄ in the medium is about 1 mM, about 2 mM, about 3 mM.

In some embodiments, the concentration of ferric citrate in the medium is from about 0 to about 0.150 mM. In yet other embodiments, the concentration of ferric citrate in the medium is from about 0.035 mM to about 0.1 mM. In other embodiments, the concentration of ferric citrate in the medium is about 0.010, about 0.020, about 0.030 mM, about 0.035 mM, about 0.040 mM, about 0.045 mM, about 0.050 mM, about 0.055 mM, about 0.060 mM, about 0.065 mM, about 0.070 mM, about 0.075 mM, about 0.080 mM, about 0.085 mM, about 0.090 mM, about 0.095 mM, about 0.100 mM, about 0.110 mM, about 0.120 mM, about 0.130 mM, about 0.140 mM or about 0.150 mM.

In some embodiments, the concentration of glucose in the medium is from about 0 to about 55 mM. In other embodiments, the concentration of glucose in the medium is from from about 5 mM to about 55 mM, from about 10 mM to about 50 mM, from about 10 mM to about 45 mM, from about 20 mM to about 30 mM, from about 20 mM to about 25 mM. In yet other embodiments, the concentration of glucose in the medium is about 5 mM, about 10 mM, about 15 mM, about 20 mM, about 25 mM, about 30 mM, about 35 mM, about 40 mM, about 45 mM, about 50 mM, about 55 mM.

In some embodiments, the concentration of L-methionine in the medium is from about 0 to about 67 mM. In other embodiments, the concentration of L-methionine in the medium is about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM or about 70 mM.

In some embodiments, the concentration of L-glutamic acid in the medium is from about 0 to about 55 mM. In some embodiments, the concentration of L-glutamic acid in the medium is from about 20 mM to about 50 mM. In other embodiments, the concentration of L-glutamic acid in the medium is about 1 mM, about 5 mM, about 10 mM, about 15 mM, about 20 mM, about 25 mM, about 30 mM, about 35 mM, about 40 mM, about 45 mM, about 50 mM.

In accordance with the present disclosure, the mineral medium may comprise approximately 5 mM K₂HPO₄, approximately 5 mM NaH₂PO₄, approximately 2 mM MgSO₄·H₂O, approximately 50 mM Tris, 0.02 mM ferric citrate, approximately 12.5 mM glycine, approximately 5 mM L-methionine and approximately 20 mM L-glutamic acid.

In accordance with the present disclosure, the mineral medium may comprise approximately 22 mM KH₂PO₄, approximately 48 mM Na₂HPO₄, approximately 2 mM MgSO₄·7H₂O, approximately 0.1 mM NaCl, approximately 50 mM Tris, approximately 6.70 mM L-methionine, approximately 22 mM glucose, approximately 20 mM L-glutamic acid, approximately 0.02 mM ferric citrate and glycine at a concentration of from about 1 g/L to about 10 g/L.

In accordance with the present disclosure, the mineral medium may comprise approximately 22 mM KH₂PO₄, approximately 48 mM Na₂HPO₄, approximately 2 mM MgSO₄·7H₂O, approximately 0.1 mM NaCl, approximately 50 mM Tris, 6.70 mM L-methionine, approximately 22 mM glucose, approximately 20 mM L-glutamic acid and approximately 0.02 mM ferric citrate and glycine at a concentration of approximately 5 g/L.

The presence of pulp does not appear to affect the production of cyanide. The Pseudomonas bacteria of the present disclosure may therefore produce cyanide in the presence or absence of pulp.

A person skilled in the art will also understand that the cyanide-containing solution resulting from growing or maintaining the Pseudomonas bacteria of the present disclosure may be used in the cyanide leaching process. The cyanide-containing solution thus obtained is added to the ore, concentrate of waste material and the process is carried out.

As such, the present disclosure provides a cyanide-containing solution obtained from the bacteria described herein. The cyanide-containing solution may contain bacteria (live or inactivated) or may be substantially-free of bacteria. In some embodiments, the bacteria is removed from the cyanide-containing solution and the solution used in the cyanidation process.

The cyanide-containing solution described herein may therefore be used as a lixiviant in a cyanide leaching process.

In some embodiments, the bacteria may produce cyanide at a concentration of at least 10 mg/L, at least 15 mg/L or higher than 15 mg/L.

### Source of Material

The starting material used in the leaching process is any material that potentially contains precious metals.

In exemplary embodiments, the source of material is ores. In a particular embodiment, the source of material is gold ores. In exemplary embodiments, the gold ore is non-refractory gold ore or pre-treated refractory gold ore.

In another exemplary embodiment, the source of material is a concentrate. In an exemplary embodiment, the concentrate contains gold.

In another exemplary embodiment, the source of material is waste material. An exemplary embodiment of waste material is electronic waste.

In some embodiments, the concentration of gold in the starting material may be for example, at least 0.5 g per metric ton (t), at least 1.75 g/t, at least 1.0 g/t, at least 1.25 g/t, at least 1.5 g/t, at least 2.0 g/t.

In some embodiments, the concentration of silver in the starting material may be for example, at least 0.5 g/t, at least 1.75 g/t, at least 1.0 g/t, at least 1.25 g/t, at least 1.5 g/t, at least 2.0 g/t.

### Process for Leaching Precious Metals

The present disclosure also relates to a process using the Pseudomonas bacteria disclosed herein or a preparation thereof as a lixiviant. The process may be used for leaching precious metals from ore, concentrate or waste material.

The present disclosure more particularly provides a process for obtaining precious metals from ore, concentrate or waste material that comprises contacting the ore, concentrate or waste material with the Pseudomonas bacteria disclosed herein or with a preparation thereof and isolating the precious metal. The process may be performed, for example, under conditions suitable for production of cyanide by the bacteria.

The present disclosure also relates to a process for obtaining precious metals from ore, concentrate or waste material that comprises contacting the ore, concentrate or waste material with a cyanide-containing solution obtained from Pseudomonas bacteria disclosed herein and isolating the precious metal.

The metal is isolated from a pregnant solution or slurry using methods known to a person skilled in the art.

In exemplary embodiments, the process is carried out so as to obtain precious metals.

In exemplary embodiments, the process is carried out so as to obtain a metal that is able to form a complex with cyanide ions.

In exemplary embodiments, the process is carried out so as to obtain gold.

In exemplary embodiments, the process is carried out so as to obtain silver.

For example, the material (such as ore) is crushed and/or milled, incorporated into a tank, reactor, leach pad, or other vessels in an aqueous medium. In some instances, agitation may be necessary to obtain a slurry or pulp. The Pseudomonas bacteria or preparation of the present disclosure is added and the conditions suitable for producing cyanide are provided. For example, the components of the medium, pH, salt oxygenation, temperature or else are adjusted and the process is carried out.

Alternatively, the material (such as ore) is crushed and/or milled, incorporated into a tank, reactor, leach pad, or other vessels in a cyanide-containing solution produced from the Pseudomonas bacteria of the present disclosure.

The precious metal is recovered using methods known to a person skilled in the art including methods used for conventional leaching processes.

In some embodiments, the process is a batch process. In a batch process, essentially all nutrients are provided at the beginning of the process. However, control elements such as gas, acids and bases may be adjusted.

In other embodiments, the process is a fed-batch process. In a fed-batch process, nutrients are constantly supplied so as to achieve maximum growth and production of cyanide.

In other embodiments, the process is a continuous culture process. In some instances, fresh culture medium is added to the culture so as to replace medium deprived of nutrients and/or to maintain cyanide at a concentration that does not induce its degradation.

Accordingly, in some embodiments, bacterial nutrients may be added to the medium at the start of the process. However, nutrients may be gradually added to the medium. In some embodiments, at least a quantity of the nutrients is added at the beginning of the process. In other embodiments, at least a quantity of nutrients is added during the process.

Accordingly, in some embodiments, glycine may be added to the medium at the start of the process. Alternatively, glycine may be gradually added to the medium. For example, at least a quantity of glycine is added at the beginning of the process. In other example, at least a quantity of glycine is added during the process.

In some embodiments, the process is performed at a pulp density of from about 1% to at least about 20%. For example, the process may be performed at a pulp density of from about 1% to about 20%. In another example, the process may be performed at a pulp density of from about 1% to about 15%. In yet another example, the process may be performed at a pulp density of from about 1% to about 10%. In another example, the process may be performed at a pulp density of from about 1% to about 5%.

In exemplary embodiments, the process is carried out at pulp density of approximately 1%, approximately 2%, approximately 3%, approximately 4%, approximately 5%, approximately 6%, approximately 7%, approximately 8%, approximately 9%, approximately 10%, approximately 11%, approximately 12%, approximately 13%, approximately 14%, approximately 15%, approximately 16%, approximately 17%, approximately 18%, approximately 19%, approximately 20%,

In other exemplary embodiments, the process is carried out at pulp density of from about 1% to about 20% and of at least 5%, at least 6%, of at least 7%, at least 8%, at least 9%, at least 10%, at least 11%, at least 12%, at least 13%, at least 14%, at least 15%, at least 16%, at least 17%, at least 18%, at least 19%.

In some embodiments, the process is performed with ore concentration ranging from about 1 g/L to at least about 200 g/L. In other exemplary embodiments, the ore concentration is from about 10 g/L to at least about 200 g/L. In yet other exemplary embodiments, the ore concentration is from about 50 g/L to at least about 200 g/L. In further exemplary embodiments, the ore concentration is from about 100 g/L to at least about 200 g/L. In yet further exemplary embodiments, the ore concentration is from about 150 g/L to at least about 200 g/L.

In some embodiments, the ore concentration is at least 100 g/L. In other embodiments, the ore concentration is at least 200 g/L.

In some embodiments, the process of the present disclosure the process is carried out during the growth phase of the bacteria.

In some embodiments, the process of the present disclosure the process is carried out during cyanide production by the bacteria.

In some embodiments, the process of the present disclosure the process is carried out until at least maximum cyanide production is achieved.

If desired, the process may comprise a step of removing the bacteria once maximum concentration of cyanide is obtained so as to avoid degradation of cyanide.

The process may be carried over a period ranging from one day to several weeks to several months. In some embodiments, the process is carried out over a period of one week or less such as between one to six days, between one to five days, between one to four days.

The Pseudomonas bacteria disclosed herein, or preparation thereof may be used in an in situ leaching process that involves directly leaching metals from ores without the need to excavate the ore and pumping the leached material to the surface.

The present disclosure also provides a product obtained by the process disclosed herein. In some embodiments, the product comprises precious metals such as for example and without limitations, gold and/or silver.

### Kits and Equipment

The present disclosure also relates to a kit comprising one or more containers comprising the Pseudomonas bacteria described herein. Instructions for amplifying, maintaining and/or using the bacteria may also be included.

In some embodiment, the kit comprises one or more containers for the Pseudomonas bacteria described herein and one or more containers comprising growth medium (or separate components for making growth medium).

In some embodiment, the kit comprises one or more containers for the Pseudomonas bacteria described herein and one or more containers comprising medium suitable for production of cyanide (or separate components for making same) by the bacteria.

The present disclosure also relates to a piece of equipment for mining containing the Pseudomonas bacteria described or the cyanide-containing solution described herein.

### EXAMPLES

### Example 1: Isolation of Cyanogenic Bacteria

Fifty grams of a mix of gold mine samples were enriched in 100 ml of a mineral medium containing glycine (12.5 mM) as the substrate for cyanide production at two initial pHs: pH 8.0 (slightly alkaline) and pH 10.0 (strongly alkaline). The mineral medium composition was 5 mM K₂HPO₄, 5 mM NaH₂PO₄, 2 mM MgSO₄·H₂O, 50 mM Tris, 0.02 mM ferric citrate, and was supplemented with 12.5 mM glycine, 5 mM L-methionine and 20 mM L-glutamic acid. Incubation was conducted at room temperature (22±2° C.) under agitation (150 rpm). After 5 transfers, the enrichment cultures were spread on agar culture medium (R2A agar) and the agar plates were incubated for 5 days. Morphologically distinct colonies were purified by re-streaking three times on R2A agar.

Bacterial isolates were screened for their ability to produce cyanide. Tests use the 0.5% picrate 2% Na₂CO₃ technique in which color change from yellow to orange/red is indicative of HCN production.

More particularly, production of cyanide by the bacterial isolates was screened on Petri dish by the change in color of a blotting paper (Whatman) soaked with 0.5% picrate/2% Na₂CO₃. The soaked paper was fixed inside the Petri dish containing the culture medium (R2A) at pH 7 (to allow volatilization of HCN) and incubated for 5 days. HCN-positive isolates were identified by the orange/red color of the blotting paper. On the 70 bacteria isolated, one strain scored as HCN-positive (named AE-27).

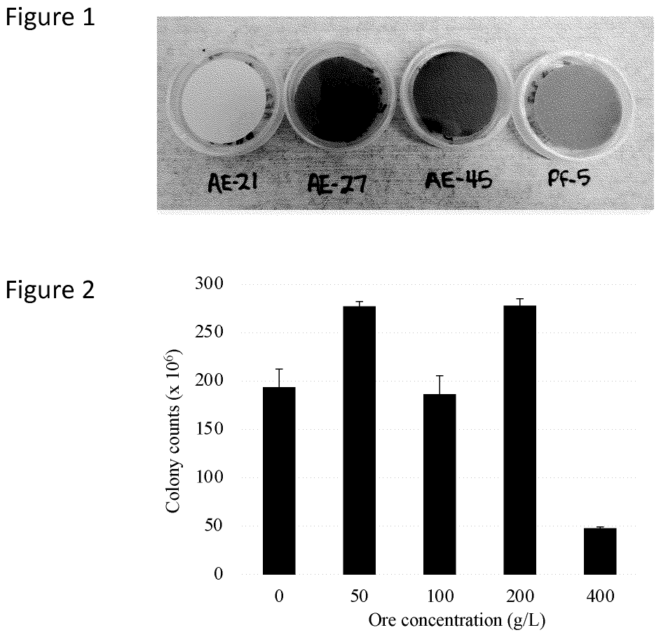

Pseudomonas protegens Pf-5 (ATCC BAA477), a known cyanogenic strain, was used as a positive control. Isolate AE-21 showed a negative reaction while strain AE-27 showed a positive reaction indicative of HCN production (FIG. 1).

### Example 2: Amplification and Maintenance

AE-27 was routinely grown in LB liquid medium (tryptone 10 g, yeast extract 5 g, sodium chloride 10 g, pH 7.2) at 25° C. and was maintained on LB agar. AE-27 was preserved in frozen stocks of 15% glycerol and stored at −80° C.

### Example 3: Characterization of the Cyanogenic Bacteria

The 16S rRNA gene was amplified for AE-27 and submitted to Sanger sequencing. The hcnC gene was amplified using the PCR primers and PCR conditions as in Dürr et al. (2015).

The 16S rRNA sequence of strain AE-27 showed 98.85% DNA (345/349) identity to Pseudomonas sp. (Blastn, GenBank). The hcnC gene sequence of AE-27 was amplified and sequenced.

The AE-27 isolate and strain Pf-5, used as a reference, were tested for their capacity to grow over a range of temperatures (4, 10, 15, 20, 25, 30, 37 and 40° C.), pH values (pH 5.0-11.0) and in the presence of various concentrations of sodium chloride (0.0, 2.0, 3.0, 4.0, 5.0, 6.0, 7.0, 10.0% w/v) using LB as the medium. The strains were incubated at room temperature (RT), except for the test at different temperatures.

### Temperature Range

Strain AE-27 and strain Pf-5, used as a reference, were tested for their capacity to grow over a range of temperatures (4, 10, 15, 20, 25, 30, 37 and 40° C.) using LB as the culture medium. Strains AE-27 and Pf-5 grew well at temperatures from 10° C. to 37° C.; also grew at 4° C. (incubated in the fridge).

### Tolerance to pH

Strain AE-27 and strain Pf-5, used as a reference, were tested for their capacity to grow over a range of pH values, from pH 5.0 to 11.0 (e.g., including 5.0, 5.5, 6.0, 6.5, 7.0, 7.5, 8.0, 8.5, 9.0, 9.5, 10.0, 10.5 and 11.0). Strain AE-27 grew at pH up to 9.5, while strain Pf-5 did not grow above 8.5.

### Salinity

Strain AE-27 and strain Pf-5, used as a reference, were tested for their capacity to grow over a range of sodium chloride concentrations (0.0, 2.0, 3.0, 4.0, 5.0, 6.0, 7.0, 10.0% w/v). Pf-5 grew at a salinity of 0 to 5% NaCl, while AE-27 grew at salinity up to 6% NaCl.

Thus, the AE-27 isolate displayed growth inside a broader range of environmental parameters, and importantly, at higher pH than the reference strain Pf-5.

### Ore Concentration

Tolerance of AE-27 to different ore concentrations was evaluated. The chemical composition and mineral composition of the ore is illustrated in Table 1 and 2 respectively. The sample contained 1.67 g/t Au.

TABLE 1
Chemical composition of the granulometric
fractions of the T2278 composite sample

| Fraction | +106 μm | −106 + 75 μm | −75 μm | Total |
|---|---|---|---|---|
| Mass (g) | 49.77 | 33.19 | 178.26 | 261.22 |
| Mass (%) | 19.1 | 12.7 | 68.2 | 100.0 |
| SiO₂ | 60.10 | 64.10 | 64.30 | 59.90 |
| Al₂O₃ | 16.00 | 18.30 | 17.00 | 15.80 |
| MgO | 2.72 | 1.51 | 1.93 | 3.26 |
| CaO | 5.52 | 3.68 | 3.68 | 6.28 |
| K₂O | 0.59 | 0.75 | 0.61 | 0.54 |
| TiO₂ | 0.42 | 0.29 | 0.33 | 0.46 |
| MnO | <0.1 | <0.1 | <0.1 | <0.1 |
| P₂O₅ | <0.1 | <0.1 | <0.1 | 0.13 |
| Co | <0.2 | <0.2 | <0.2 | <0.2 |
| Cr | <0.03 | <0.03 | <0.03 | <0.03 |
| Cu | <0.2 | <0.2 | <0.2 | <0.2 |
| Fe | 2.66 | 1.85 | 2.30 | 3.03 |
| Ni | <0.02 | <0.02 | <0.02 | <0.02 |
| Pb | <0.02 | <0.02 | <0.02 | <0.02 |
| S | 0.60 | 0.40 | 0.50 | 0.50 |
| Zn | <0.02 | <0.02 | <0.02 | <0.02 |

TABLE 2
Mineral composition of the granulometric
fractions of the T2278 composite sample

| Fraction | +106 μm | −106 + 75 μm | −75 μm | Total |
|---|---|---|---|---|
| Mass (g) | 49.77 | 33.19 | 178.26 | 261.22 |
| Mass (%) | 19.1 | 12.7 | 68.2 | 100.0 |
| Quartz | 55.6 | 59.0 | 55.2 | 55.8 |
| Albite | 22.3 | 18.1 | 13.3 | 15.7 |
| Chlorite | 2.9 | 4.3 | 8.4 | 6.8 |
| Muscovite | 8.1 | 5.2 | 3.6 | 4.7 |
| Hornblende | 1.9 | 2.6 | 4.0 | 3.4 |
| Calcite | 1.0 | 1.6 | 3.9 | 3.0 |
| Biotite | 1.1 | 1.6 | 2.2 | 1.9 |
| Pyrite | 1.0 | 0.9 | 0.8 | 0.9 |
| Hematite | 0.20 | 0.25 | 0.76 | 0.59 |
| Gypsum | 0.1 | 0.2 | 0.6 | 0.4 |
| Plagioclase | 0.71 | 0.45 | 0.32 | 0.41 |
| Epidote | 2.92 | 2.70 | 2.61 | 2.69 |
| Apatite | 0.08 | 0.17 | 0.36 | 0.28 |
| Ankerite | 0.04 | 0.18 | 0.31 | 0.24 |
| Titanite | 0.14 | 0.21 | 0.27 | 0.24 |
| Rutile | 0.12 | 0.10 | 0.17 | 0.15 |
| Orthoclase | 0.14 | 0.12 | 0.11 | 0.12 |
| Other silicates | 1.6 | 2.5 | 3.1 | 2.7 |
| Total | 100 | 100 | 100 | 100 |

Since the purpose of this study is to use bacterium AE-27 to dissolve gold, their tolerance to high concentrations of this sample was evaluated. Strain AE-27 was inoculated at an optical density at 600 nm (OD₆₀₀) of 0.03 in a mineral medium at pH 9.0 with ore concentrations from 0 g/L to 500 g/L, in triplicate, in Erlenmeyer flasks. The cultures were incubated at room temperature at 150 rpm for 2 days. Dilutions of 10⁻⁶ were spread on R2A agar and the number of colonies (expressed as colony forming unit, CFU) was counted after 5 days.

Results demonstrated that the T2278 composite sample did not inhibit growth of AE-27 up to 200 g/L, but inhibition was observed at 400 g/L (FIG. 2).

### Example 4: Optimization of Cyanide Production

### Culture Medium and Quantification of Cyanide.

Bacterial strains were grown in 2-L Erlenmeyer flasks in a modified glycine medium (referred to herein as “CN medium”) composed of 22 mM KH₂PO₄, 48 mM Na₂HPO₄, 2 mM MgSO₄·7H₂O, 0.1 mM NaCl, 50 mM Tris, 6.70 mM L-methionine, 22 mM glucose, 20 mM L-glutamic acid and 0.02 mM ferric citrate. Glycine was tested at different concentrations (such as 0, 2.5, 5.0, 7.5, 10.0 g/L) with one of the preferred glycine concentration for use in the CN medium being 5 g/L. Bacterial growth was monitored by measuring the optical density at 600 nm (OD₆₀₀) in a UV-Vis spectrophotometer. Free cyanide was quantified by the barbituric acid/isonicotinic acid colorimetric method described by Nagashima and Ozawa (1981).

### Cyanide Production as a Function of the Growth Phase

Strains AE-27 and Pf-5 were grown in the CN medium containing 5 g/L of glycine and incubated at 25° C. in agitation (150 rpm). A pH of 8.5 was selected because it is the maximal pH for growth of Pf-5.

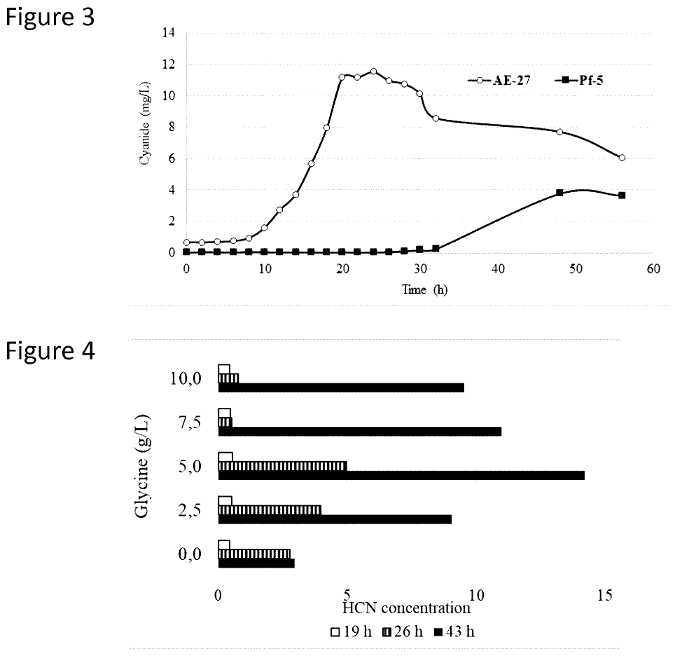

The kinetics of cyanide production on a period of 56 hours for strains AE-27 and Pf-5 at pH 8.5, are reported in FIG. 3.

As shown in FIG. 3, the reference bacterium, Pf-5, produced a maximum of 3.8 mg CN/L while strain AE-27 produced 11.5 mg CN/L. Cyanide reached its maximal concentration around 21 h and 48 h of incubation, respectively, for AE-27 and Pf-5. The concentration of cyanide in solution decreased after 48 h of incubation for AE-27.

### Example 5: Optimization of Parameters

Optimization of cyanide production by strain AE-27 was done for the following parameters: (1) glycine concentration, (2) pH, (3) pulp density and (4) temperature. For each parameter, experiments were carried out in triplicate and with negative controls (without bacteria).

### Glycine Concentration

Strain AE-27 was incubated in the CN medium containing different concentrations of glycine, i.e., 0 g/L, 2.5 g/L, 5.0 g/L, 7.5 g/L and 10.0 g/L at 2% pulp density at 25° C. The highest cyanide concentration (14.23 mg CN/L) was detected after 43 h of incubation in the culture medium containing 5.0 g/L glycine (FIG. 4). Therefore, the optimal glycine concentration for cyanide production by AE-27 was established at 5.0 g/L and used for the next optimization experiments.

pH

Strain AE-27 was incubated in the CN medium at three different pH values (9.0, 9.5 and 10.0) at 2% pulp density at 25° C.

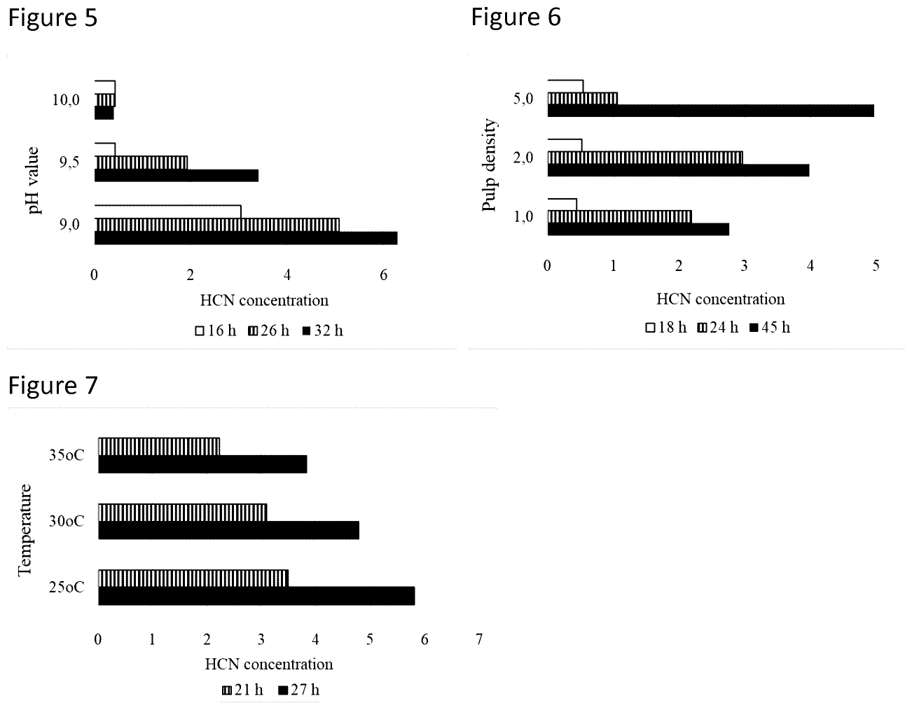

Maximum cyanide production was obtained at pH 9.0 and correlated with bacterial growth (FIG. 5). The bacterium barely grows at pH 10. The optimal pH value for cyanide production was determined to be pH 9.0 and this pH was used for the next optimization experiments.

### Pulp Density

Strain AE-27 was incubated in the CN medium at different pulp density (1%, 2% and 5%) at pH 9.0 and at 25° C.

Cyanide production was not significantly affected by an increase in pulp density from 1% to 5%. The maximum cyanide concentration was detected after 24 h in the solution at 5% pulp density (16.80 mg CN/L), which is not significantly different from the cyanide concentration at 1% pulp density (15.56 mg CN/L). Higher pulp densities (>20%) are tested (FIG. 6).

### Temperature

AE-27 was incubated in the CN medium at pH 9.0 and at different temperatures at 2% pulp density.

Pseudomonas AE-27 is a mesophilic strain (section 1.3) and cyanide production was not significantly affected in the temperature range from 25° C. to 35° C. (FIG. 7). A broader range of temperature is tested, including for example, from about 4° C. and up to about 35° C.

### Example 6: Biocyanidation Assays

Biocyanidation assays were conducted to evaluate the efficacy of the cyanogenic bacterium AE-27 to solubilize gold from the T2278 sample. In each assay, AE-27 was inoculated, from an overnight culture, in the CN medium at an OD₆₀₀ of 0.03. Erlenmeyer flasks were incubated at room temperature with agitation at 150 rpm for 48 h and 120 h. A control without bacteria was run in parallel. At the end of the incubation period, the bacterial solution was centrifuged at 6,000×g and filtered through a 0.02 μm syringe filter. The remaining solid was washed with distilled water and dried at 105° C. for 18 h. Samples were prepared and analyzed by inductively coupled plasma mass spectrometry (ICP-MS).

Strain AE-27 successfully solubilized 14% of gold in the sample after 48 h of incubation at 5% pulp density.

Since silver is able to complex with cyanide, it is expected that strain AE-27 will also be able to leach silver from ore, concentrate or waste material.

In summary, among the 70 bacterial isolates screened, one was shown to produce cyanide (isolate AE-27). The strain was phylogenetically closely related to Pseudomonas putida. AE-27 grew at pH up to 9.5, at temperature up to 37° C., and salt concentration (NaCl) up to 6%, which was slightly better than what was observed for a known cyanogenic strain, Pseudomonas protegens Pf-5 (ATCC BAA477), which did not grow at pH 9.0 and above. Strain AE-27 could grow in the presence of high ore concentrations, with no observable toxicity at the highest concentration tested (300 g/L). Parameters that can influence cyanide production by bacteria were evaluated (glycine concentration in the culture medium, pH, pulp density, temperature). Results showed an optimal glycine concentration of 5 g/L, pH of 9.0, and temperature of 25° C. Production of cyanide did not vary significantly as a function of pulp density. AE-27 could produce 16 mg free CN/L at pH 9.0. Gold extraction by AE-27 reached 14% after 48 h of incubation at 5% pulp density at pH 9.0 at room temperature (˜22° C.). Gold in solution was below the ICP-MS detection limit when incubation was prolonged to 120 h in an attempt to solubilize more gold. We suspect that this is due to biodegradation of the cyanide-Au complex. Overall, the results showed that bacteria isolated as part of this project successfully dissolved gold from a non-refractory composite ore sample, at relatively high pH without any adaptation stage. Efforts should now focus to extend the efficient biocyanidation period over 48 h.

### Example 7: Leaching of Precious Metals

The AE-27 strain is provided to the user as a lyophilized or frozen material, in solution or else as may be required.

The AE-27 strain is contacted with material containing precious metals such as gold and/or silver, including for example and without limitation, ore, concentrate, waste material (electronic, tailing etc.) and the like.

The starting material may be, for example and without limitations, non-refractory or refractory gold ore or concentrate.

In the case of non-refractory gold ore, the AE-27 may be included in the extraction process as a replacement of or supplement to cyanide (e.g., NaCN, KCN, CaCN₂). For example, the ore is crushed and/or milled, incorporated into a tank, reactor, leach pad or other container and aqueous medium (water, culture medium etc.) containing the AE-27 strain is added so as to obtain a slurry or pulp.

The process of performed under conditions that allow production of cyanide by the AE-27 strain. Exemplary conditions are discussed above and include for example and without limitations, alkaline pH, glycine concentration and/or temperature.

The AE-27 stain is added to the ore or concentrate in batch or in continuous flow. Glycine may also be added gradually.

Glycine appears to be the most economical and environmentally friendly option. However, other amino acids can be used such as threonine, phenylalanine, glutamine or others.

In industrial cyanidation processes, the control of pH is usually achieved with lime, NaOH, KOH or else. Cyanidation processes also require water and oxygen or air (e.g., aeration, bubbling). The process may be carried out under agitation.

In the case of refractory gold ore or concentrate, pre-treatment (roasting, bio-oxidation, pressure oxidation, Albio process) may be beneficial. The pre-treated ore or concentrate may then be treated with the AE-27 strain as indicated above.

Instead of using the AE-27 strain per se, a person skilled in the art will understand that the culture medium that is recovered following production of cyanide by the AE-27 strain (i.e., substantially free of bacteria) may be used for leaching gold or other precious metals.

The AE-27 strain may also be used for in situ leaching (solution mining). In situ leaching involves leaving the ore in the ground, and recovering the metals from it by dissolving them and pumping the pregnant solution to the surface where the metals can be recovered.

Extracted gold can be purified or refined by methods known to a person skilled in the art including by the Miller process or Wohlwill process or else. Gold can also be recovered by CIP (carbon-in-pulp), CIL (carbon-in-leach) and CIC (carbon-in-columns).

The embodiments and examples described herein are illustrative and are not meant to limit the scope of the claims. Variations of the foregoing embodiments, including alternatives, modifications and equivalents, are intended by the inventors to be encompassed by the claims. Citations listed in the present application are incorporated herein by reference.

### REFERENCES

Castric P A. Hydrogen cyanide, a secondary metabolite of Pseudomonas aeruginosa. Canadian Journal of Microbiology. 1975 May 1; 21(5):613-8.
Castric P A. Glycine metabolism by Pseudomonas aeruginosa: hydrogen cyanide biosynthesis. Journal of Bacteriology. 1977 May; 130(2):826-31.
Dürr S, Sessitsch A, Brader G, Trognitz F. Development of high-throughput methods for the detection of hydrogen cyanide-producing bacteria for the application in biocontrol Entwicklung von Hochdurchsatzmethoden zur Detektion von Cyanwasserstoff produzierenden Bakterien für die Anwendung in der Biokontrolle. Tagungsbericht 2015. 2015:23.
Nagashima, S. and Ozawa, T., 1981. Spectrophotometric determination of cyanide with isonicotinic acid and barbituric acid. International Journal of Environmental Analytical Chemistry, 10(2), pp. 99-106.

1-2. (canceled)

3. An alkali-tolerant Pseudomonas bacteria characterized by an endogenous 16S rRNA sequence that is at least 95% identical to the sequence set forth in SEQ ID NO: 1, wherein the bacteria has the ability to produce cyanide.

4. Pseudomonas bacteria strain AE-27 as defined in International Depositary Authority of Canada Accession Number 160322-01.

5-9. (canceled)

10. A process for obtaining at least one precious metal from ore, concentrate or waste material, the process comprising:
(a) contacting the ore, concentrate, or waste material with the Pseudomonas bacteria of claim 4, or with a preparation thereof, under conditions allowing production of cyanide by the bacteria; and
(b) isolating the at least one precious metal.

11. A process for obtaining at least one precious metal from ore, concentrate or waste material, the process comprising:
(a) contacting the ore, concentrate or waste material with the Pseudomonas bacteria of claim 3 or with a preparation thereof, under conditions allowing production of cyanide by the bacteria; and
(b) isolating the at least one precious metal.

12. The process of claim 11, wherein the at least one precious metal is gold and/or silver.

13. The process of claim 11, wherein the process is performed at an alkaline pH.

14. The process of claim 11, wherein the pH is between 8.0 and 10.0.

15. The process as defined in claim 13, wherein the pH is between 8.5 and 9.5.

16. (canceled)

17. The process of claim 11, wherein the process is performed at a temperature of between 4° C.±2° C. to 35° C.±2° C.

18. The process of claim 17, wherein the process is performed at a temperature of between 25° C.±2° C. to 35° C.±2° C.

19. (canceled)

20. The process of claim 11, wherein the process is performed in a medium comprising: a substrate for the hydrogen cyanide synthase enzyme, nutrients, a source of energy, and/or minerals.

21. The process of claim 11, wherein the process is performed in a mineral medium.

22. The process of claim 21, wherein the mineral medium comprises potassium, sodium, magnesium, iron and glycine, or equivalents thereof.

23. The process of claim 20, wherein the substrate is glycine.

24. The process of claim 23, wherein the glycine is at a concentration of from about 1 g/L to about 10 g/L.

25. (canceled)

26. The process of claim 23, wherein a quantity of the glycine is gradually added to the mineral medium.

27. The process of claim 11, wherein the process is performed at a pulp density of from about 1% to at least about 20%.

28. The process of claim 11, wherein the process is performed in a mineral medium comprising glycine at a concentration of approximately 5 g/L, at a pH of approximately 9.0, and at a temperature of approximately 25° C.

29. The process of claim 11, wherein the process is performed during the growth phase of the bacteria.

30-37. (canceled)

38. A piece of equipment for mining containing the Pseudomonas bacteria of claim 3.

39. (canceled)
